(19) United States
(12) Patent Application Publication
(10) Pub. No.: US 2007/0256691 A1
(43) Pub. Date: Nov. 8, 2007

(54) Conception aid

(76) Inventors: Mark Ellery Ogram, Tucson, AZ (US); Karen Lee Ogram, Tucson, AZ (US)

Correspondence Address: Mark E. Ogram, # 203, 7454 E. Broadway, Tucson, AZ, 85710, US

(21) Appl. No.: 11/407,403

(22) Filed: Apr. 19, 2006

(51) Int. Cl. A61F 6/06; A61F 6/02

(52) U.S. Cl. 128/830; 128/837; 128/842

(57) ABSTRACT

A conception aid which places a treatment for the sperm within the vagina of the female. The treatment creates either an alkaline or an acidic environment through which the sperm must pass. This environment encourages either the Y-sperm or the X-Sperm. Delivery of the treatment is through a variety of mechanisms including: specially designed condoms; special diaphragms; vaginal suppositories; and, lubricants.

### BACKGROUND OF THE INVENTION

This invention relates generally to conception and more particularly to an apparatus used to encourage the conception of a particular sex.

The sex of an unborn child has been of keen interest since Adam and Eve. Producing a girl is sought after when there are several boys in the family; a boy is important for the family name, working the land, or an heir to the throne.

In ancient times, conjurers were used and a great variety of concoctions, usually with a success rate of half the time.

With more modern scientific knowledge, it is now commonly known that it is the sperm which determines the sex of the child. Even after the XY chromosomes were clearly known, the number of urban myths about when a boy is conceived persisted. Diet, vigor, and even the power of suggestion are widely believed as being at least partially responsible for whether a girl is conceived or a boy is.

A true scientific approach was made by Landrum B. Shettles and was published in How to Choose the Sex of your Baby (Broadway Books, 1540 Broadway, New York, N.Y. 10036, copyright 1970, 1977, 1984, 1989, and 1999), incorporated hereinto by reference.

The Shettles technique involves a close monitoring of the female's ovulation cycle together with the use of alkaline douches to encourage the conception of a boy and an acidic douche to encourage the conception of a girl.

Unfortunately, the use of a douche immediately prior to intercourse is far from “romantic”; and, as such, tends to discourage its use.

It is clear there is a need for an easy to use apparatus which will encourage the conception of a selected sex.

### SUMMARY OF THE INVENTION

The present invention provides for an easily applied mechanism which encourages the conception of a specific sex. To this end, the invention provides a conception aid which has a carrier adapted to be inserted into a vagina together with a chosen treatment which has been secured to the carrier.

In practice, two different combinations are available for use, one to encourage the conception of a boy and a different one used to encourage the conception of a girl. The couple, by using one of the combinations, is exercising a choice on the sex conceived.

The treatment which is used to encourage the conception of a specific sex, is delivered to the vagina via a carrier. A variety of carriers are applicable to this invention and include: specially designed condoms; special diaphragms; vaginal suppositories; and, lubricants.

The condom embodiment is dramatically different than a typical condom in that the condom of this invention is not intended to serve as a barrier to prevent the sperm from impregnating the female; rather, this condom has two openings: one for insertion of the penis and the second opening for the ejaculated sperm to exit into the vagina.

In similar fashion, the diaphragm is also the anti-thesis of a standard diaphragm. In this invention, the diaphragm is not used to provide a barrier, rather the diaphragm is used to create the desired environment to encourage a specific type of sperm (X or Y) to survive to fertilization of the female's egg.

While the terms “condom” and “diaphragm” are used in this discussion, the structures of these terms are not intended to establish a barrier for the sperm, rather, these instruments are used to create a desired environment while permitting the sperm to travel to the egg.

The suppository and the lubricant embodiments of this invention are used as carriers of the treatment. The treatment, as described below, is mixed into the body of the suppository or the lubricant.

The carriers are used to deliver the treatment into the female's vagina. The treatment is chosen to encourage either the Y-laden sperm or the X-laden sperm by creating an environment through which a selected sperm is encouraged while the other type of sperm is discouraged. This is done by creating a pH which is either alkaline or acidic.

Those of ordinary skill in the art readily recognize a variety of techniques which will produce these environments such as the use of baking soda (for alkaline to encourage the Y-sperm) or powered citric acid, aspirin/acetylksalicylic acid, or vinegar (for acidic environments to encourage the X-sperm).

In this way, the treatment creates a micro environment within the female's vagina which discourages the not-sought-after sperm type, thereby creating a better chance for the sought-after sperm type.

The invention, together with various embodiments thereof will be more fully explained by the accompanying drawings and the following descriptions thereof.

### DRAWINGS IN BRIEF

FIG. 1 illustrates the preferred embodiment of the invention.

FIG. 2 illustrates an embodiment of the invention which operates like a diaphragm.

FIG. 3 illustrates an embodiment of the invention which is used as a vaginal suppository.

FIG. 4 illustrates an embodiment of the invention in which the treatment is mixed with a lubricant in either a gel or foam form.

### DRAWINGS IN DETAIL

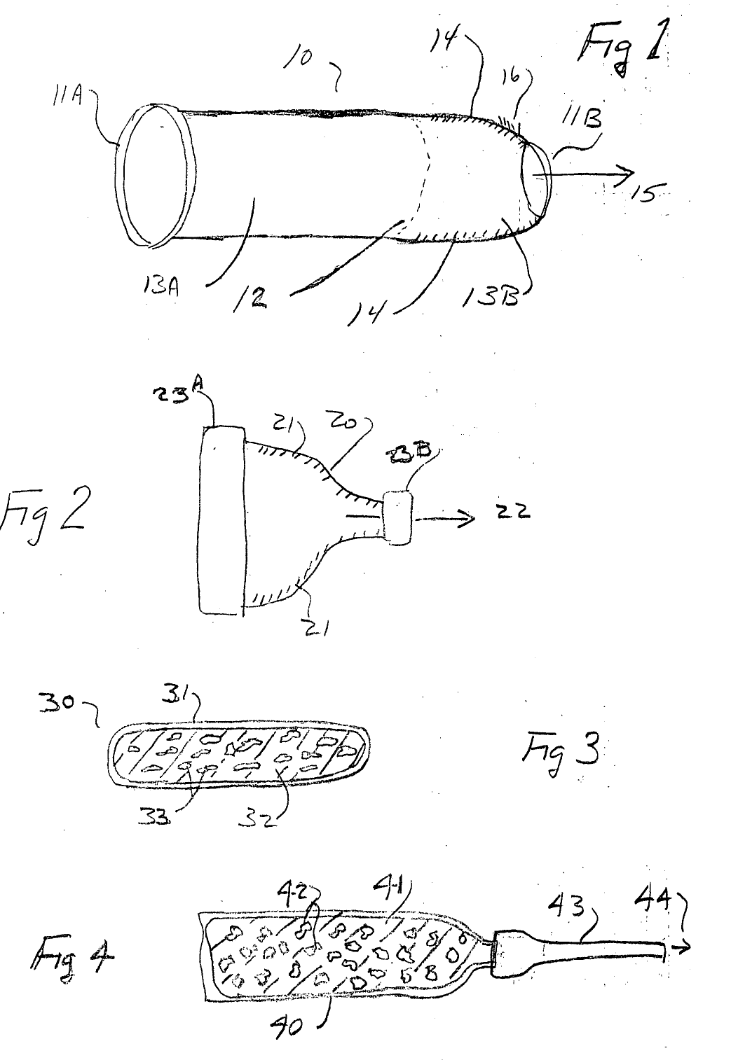

FIG. 1 illustrates the preferred embodiment of the invention.

As used within this discussion, the term “condom” means a sheath which fits over the penis. The condom of this discussion is not intended to prevent conception but rather is a conception aid.

Condom 10 has open end 11A and open end 11B. The male's penis is placed within condom 10 via open end 11A. The length of condom 10 is chosen so that the penis does not fully fill condom 10. As this illustration shows, the penis extends to a location 12, thereby dividing the interior of condom 10 into section 13A (which sheaths the penis) and section 113B.

Section 13B is coated on an interior portion with a treatment 14. During intercourse, the ejaculate is subjected to treatment 14 prior to escaping via open end 11B as illustrated by arrow 15. Treatment 14 is chosen to encourage the survival of a specific sperm (alkaline to encourage the Y sperm; acidic to encourage the X sperm).

While the use of baking powder is the preferred treatment 14 for an alkaline environment within section 13, those of ordinary skill in the art readily recognize a variety of other chemicals which will produce the sought after alkaline environment. Similarly, while the preferred treatment 14 for an acidic environment is either vinegar or citric acid, those of ordinary skill in the art readily recognize other chemicals which serve this purpose.

In an alternative embodiment, treatment 16 is placed on the outside of condom 10 to provide an enhanced environment for treating the sperm.

While the manufacture of condom 10 is obvious to those of ordinary skill in the art, a variety of manufacturing processes and shapes are well known, including, but not limited to: U.S. Pat. No. 6,983,751, issued to Osterberg on Jan. 10, 2006, and entitled “Condom”; U.S. Pat. No. 6,913,018, issued to Van Rensburg on Jul. 5, 2005, and entitled “Condom Donning Device”; U.S. Pat. No. 6,895,567, issued to Praml on May 24, 2005, and entitled “Condom with Inflatable Portion”; U.S. Pat. No. 6,892,732, issued to Wang et al. On May 17, 2005, and entitled “Condom Coated with Lactic Acid Bacteria Contained Composition”; U.S. Pat. No. 6,840,244, issued to Kemp, on Jan. 11, 2005, and entitled “Condom with an Erectogenic Composition”; U.S. Pat. No. 6,732,736, issued on May 11, 2004, and entitled, “Condom with Applicator”; and, U.S. Pat. No. 6,718,983, issued to Suzuki on Apr. 13, 2004, and entitled, “Condom”, all of which are incorporated hereinto by reference.

FIG. 2 illustrates an embodiment of the invention which operates like a diaphragm. As used herein, the term “diaphragm” means a flexible metal ring which is covered with a partially dome-shaped sheet of elastic material and which is used within the vagina. The diaphragm of this invention though is modified as indicated below and is not intended to prevent pregnancy, rather, the diaphragm of this invention is a conception aid.

Diaphragm 20 uses ring 23A to secure itself to the walls within the female's vagina. Ring 23A is open as well as ring 23B, thereby allowing the ejaculate to travel therethrough as indicated by arrow 22.

On an interior wall of diaphragm 20 is treatment 21. Treatment 21 creates an environment (alkaline or acidic) through which the sperm must travel. This environment encourages either the X-sperm or the Y-sperm.

In an alternative embodiment, ring 23B is impregnated with the desired treatment. This treatment is released on both sides of the diaphragm 20 to provide enhanced sperm type selection.

Those of ordinary skill in the art readily recognize a variety of different diaphragms which will serve this function as outlined herein, including U.S. Pat. No. 6,796,973, issued on Sep. 28, 2004, to Contente et al. And entitled “Vaginal Discharge Collection Device and Intravaginal Drug Deliver System”; and, U.S. Pat. No. 6,569,083, issued May 27, 2003, to Kassman and entitled, “Male, Hermaphroditic, and Female Condoms Exerting Lateral Pressure on the Penis and the Vagina”, both of which are incorporated hereinto by reference.

FIG. 3 illustrates an embodiment of the invention which is used as a vaginal suppository.

In this embodiment of the invention suppository 30 is configured to be inserted into the vagina prior to intercourse. Skin 31 melts or is ruptured within the vagina thereby permitting the interior contents of suppository 30 to extend within the vagina.

The interior contents of suppository 30 consist, in this embodiment, of a carrier 32 together with treatment 33. Treatment 33 is chosen to encourage conception of a particular sex as outlined above.

In some embodiments of the invention, carrier 32 is a lubricant or a stimulant chosen to enhance sensations.

One such suppository is described in U.S. Pat. No. 7,004,171, issued to Benita et al. On Feb. 28, 2006, and entitled, “System for Transvaginal Drug Deliver”, incorporated hereinto by reference.

FIG. 4 illustrates an embodiment of the invention in which the treatment is mixed with a lubricant.

In this illustration lubricant 41 has mixed with it treatment 42. This combination is contained within tube 40. Nozzle 43 permits the application of the combination within the vagina as indicated by arrow 44.

Again, treatment 42 is chosen to encourage the specific sperm type chosen by the couple.

A variety of lubricants are well known to those of ordinary skill in the art, including the lubricant described in U.S. Pat. No. 6,624,198, issued on Sep. 23, 2003, to Porat and entitled, “Aids Prophylactic Lubricating Composition and Devices for Its Use”, incorporated hereinto by reference.

It is clear that the present invention provides an apparatus that is easy to use and which encourages the conception of a selected sex.

What is claimed is:

1. A combination for encouraging the conception of a specific sex comprising:
a) a carrier adapted to be inserted into a vagina; and,
b) a treatment secured to said carrier, said treatment chosen to encourage a selected type of sperm.

2. The combination according to claim 1, wherein said treatment creates a selected pH environment through which sperm must pass.

3. The combination according to claim 2, wherein said pH environment is above 7.

4. The combination according to claim 2, wherein said pH environment is below 7.

5. The combination according to claim 2, wherein said carrier includes a sheath adapted to be placed over a penis and having a first open end and a second open end.

6. The combination according to claim 5, wherein said treatment is on an interior portion of said sheath.

7. The combination according to claim 6, wherein the treatment is further secured to an exterior surface of said sheath.

8. The combination according to claim 2, wherein said carrier includes a diaphragm configured to permit sperm to pass therethrough.

9. The combination according to claim 2, wherein said carrier includes a lubricant.

10. The combination according to claim 2, wherein said carrier includes a suppository.

11. An apparatus for encouraging conception of a specific sex comprising:
a) a carrier adapted to be inserted into a vagina; and,
b) a treatment secured to said carrier, when said carrier is inserted into said vagina, said treatment creating a selected pH environment through which sperm must pass for fertilization of an egg.

12. The apparatus according to claim 11,
a) wherein said carrier includes a condom having a first open end and a second open end; and,
b) wherein said treatment is secured to an interior portion of said condom.

13. The apparatus according to claim 11,
a) wherein said carrier includes a diaphragm configured to permit sperm to pass therethrough; and,
b) wherein said treatment is secured to a face of said diaphragm.

14. The apparatus according to claim 11,
a) wherein said carrier includes a lubricant; and,
b) wherein said treatment is mixed within said lubricant.

15. The apparatus according to claim 11,
a) wherein said carrier includes a suppository; and,
b) wherein said treatment is contained within said suppository.

16. A conception aid comprising:
a) a carrier adapted to be inserted into a vagina; and,
b) a treatment secured to said carrier, said treatment, when said carrier is placed within the vagina, creating a selected pH environment through which sperm must travel after ejaculation.

17. The conception aid according to claim 16, wherein said carrier includes a condom having a first open end and a second open end through which ejaculated sperm pass.

18. The conception aid according to claim 17, wherein said treatment is on an interior portion of said condom.

19. The conception aid according to claim 16, wherein said carrier includes a diaphragm having openings to permit sperm to pass therethrough.
